(19) United States
(12) Patent Application Publication
(10) Pub. No.: US 2006/0246021 A1
(43) Pub. Date: Nov. 2, 2006

(54) Sunless tanning composition and methods for using

(76) Inventor: James A. Roszell, Henderson, NV (US)

Correspondence Address: MERCHANT & GOULD PC, P.O. BOX 2903, MINNEAPOLIS, MN, 55402-0903, US

(21) Appl. No.: 11/120,132

(22) Filed: May 2, 2005

(51) Int. Cl. A61K 8/35; A61K 8/73

(52) U.S. Cl. 424/59; 424/70.13

(57) ABSTRACT

A sunless tanning composition is provided according to the invention. The sunless tanning composition includes an effective amount of a tanning active component to provide a darkening effect of skin tissue after exposure to the skin tissue, a polymer component, water, an effective amount of an acidifying agent to provide the composition with a pH of less than about 5, and wherein the composition has the viscosity of at least about 3,000 cSt. The polymer component can be characterized as a skin bonding polymer component to hold the tanning active component in exposure to the skin tissue when applied to the skin tissue. The polymer component can be characterized as a polymer having an average molecular weight of about 2,000 to about 500,000. A method of applying a sunless tanning composition to skin tissue is provided.

### FIELD OF THE INVENTION

The invention relates to a sunless tanning composition and to methods for using a sunless tanning composition. The sunless tanning composition includes a polymer component and a tanning active component, and can be provided in the form of a lotion for ease of application to skin tissue.

### BACKGROUND OF THE INVENTION

Sunless tanning compositions are available for application to skin tissue. In general, the sunless tanning compositions are intended to provide skin with a tanned appearance, without exposure to sun or radiant energy sources such as tanning beds and ultra-violet lamps. Sunless tanning compositions are often applied as lotions or by spray application to skin tissue.

Numerous sunless tanning compositions are described by, for example, U.S. Pat. No. 6,482,397 to Scott et al., U.S. Pat. No. 6,261,541 to Karpov et al., U.S. Pat. No. 6,231,837 to Stroud et al., U.S. Pat. No. 5,827,506 to McShane et al., U.S. Pat. No. 5,756,075 to Meyer, U.S. Pat. No. 5,750,092 to Meyer et al., and U.S. Pat. No. 6,645,822 to Meyer et al.

### SUMMARY OF THE INVENTION

A sunless tanning composition is provided according to the invention. The sunless tanning composition includes an effective amount of a tanning active component to provide a darkening effect of skin tissue after exposure to the skin tissue, a polymer component, water, an effective amount of an acidifying agent to provide the composition with a pH of less than about 5, and wherein the composition has the viscosity of at least about 3,000 cSt. The polymer component can be characterized as a skin bonding polymer component to hold the tanning active component in exposure to the skin tissue when applied to the skin tissue. The polymer component can be alternatively characterized as a polymer having an average molecular weight of about 2,000 to about 500,000.

A method of applying a sunless tanning composition to skin tissue is provided according to the invention. The method includes a step a rubbing the sunless tanning composition having a viscosity of at least about 3,000 cSt. onto skin tissue. The sunless tanning composition can remain on the skin tissue for a sufficient length of time so that the tanning active component in the sunless tanning composition can provide a darkening effect to the skin tissue.

### DETAILED DESCRIPTION OF THE INVENTION

A sunless tanning composition is provided by the invention. A sunless tanning composition is a composition that, when applied to skin tissue, results in a darkening of the skin tissue as a result of an interaction between the skin tissue and a tanning active component in the sunless tanning composition. The interaction can be a chemical reaction of the tanning active component with the skin tissue. The darkening effect can occur without exposure to ultraviolet radiation from the sun. The sunless tanning composition can be referred to more simply as the tanning composition or as the composition.

The sunless tanning composition can be provided in the form of a lotion and applied to skin tissue by rubbing the composition onto the skin tissue. The sunless tanning composition can have a viscosity that allows it to be applied to skin tissue conveniently as a lotion. The sunless tanning composition can have a viscosity that is sufficiently high so that the lotion can be applied from a container (e.g., a tube or a bottle) to a person's hand or a location on the person's body, and the lotion can be rubbed onto the skin tissue while avoiding the dripping that would be associated with a lower viscosity product. For example, the lotion can be applied to a person's hand without worrying that the lotion will run through the person's fingers before the person can apply the lotion to skin tissue. The lotion can be characterized as having a viscosity sufficient to allow it to be conveniently applied as a lotion. For example, the sunless tanning composition can have a viscosity of greater than about 3,000 cSt (centistokes). The sunless tanning composition can be referred to as a sunless tanning lotion or more simply as a lotion.

The sunless tanning lotion includes a tanning active component, a skin bonding polymer component, water, and a pH adjusting agent to provide the sunless tanning composition at a pH of less than about 5. Additional components that can be included in the sunless tanning composition include surfactant, coloring agent, preservative, thickening agent, emollient, humectant, antioxidant, fragrance, and chelating agent. The sunless tanning lotion can include any one or more of these additional components.

The sunless tanning composition can be provided as an emulsion. Exemplary types of emulsions include oil in water emulsions, and water in oil in water emulsions. In general, the water phase can be provided as the continuous phase because of the large amount of water in the sunless tanning composition.

The sunless tanning composition can be applied by hand application to skin tissue. In general, hand application refers to rubbing the sunless tanning composition onto skin tissue by use of a hand. As a result of applying the sunless tanning composition by hand application, the sunless tanning composition can have a viscosity that allows it to be applied by hand application. Sunless tanning compositions are available that are applied by spray application where the composition is applied as a mist or spray. For example, a user can enter a booth where a mist or spray of the sunless tanning composition is applied to the exposed skin tissue. An exemplary sunless tanning composition that can be applied by spray application is disclosed in U.S. application Ser. No. 10/933,128 that was filed with the United States Patent and Trademark Office on Sep. 2, 2004. U.S. application Ser. No. 10/933,128 is assigned to Skinvisible Pharmaceuticals, Inc., which is the assignee of the above-identified patent application. The entire disclosure of U.S. application Ser. No. 10/933,128 is incorporated herein by reference. In general, for a composition to be applied as a mist, the composition can be provided having a relatively low viscosity.

Skin Bonding Polymer Component

The sunless tanning composition can include a skin bonding polymer component. The skin bonding polymer component can include any polymer that, when applied to the skin, helps hold the tanning active component to the skin. The skin bonding polymer component holds the tanning active component in exposure to skin tissue when applied to the skin tissue for a sufficient length of time to allow the tanning active component to provide a darkening effect of the skin tissue. The skin bonding polymer component can be referred to a the polymer component. The polymer component can be characterized as a polymer having an average molecular weight of at least about 2,000 and as a polymer having an average molecular weight of less than about 500,000.

The polymer component can include a hydrophobic polymer/hydrophilic polymer adduct and can include water and other components. Polymer components that can be used according to the invention include the topical compositions disclosed in U.S. Pat. No. 6,756,059. The entire disclosure of U.S. Pat. No. 6,756,059 is incorporated herein by reference

The sunless tanning composition can bind or adhere to skin tissue for a length of time, and can hold or contain the tanning active component within the composition, and can keep it exposed to the skin tissue. It is expected that the sunless tanning composition is able to adhere or bind to skin tissue for at least about four hours and hold the tanning active component contained therein in proximity to skin tissue for that length of time. In general, it is expected that the sunless tanning composition will adhere the tanning active component to skin tissue for a length of time sufficient to allow the tanning active component to react with the skin tissue and provide a desired level of darkening effect. It is believed that a person can shower relatively soon after application of the sunless tanning composition, and the polymer component will hold the tanning active component to the skin to provide darkening of the skin tissue. It is understood that prior art sunless tanning compositions often require that users of the composition not shower for a period of 8 to 12 hours after application of the sunless tanning composition. By utilizing a polymer component to adhere the tanning active component to the skin tissue, it is possible to shower much sooner compared to the use of prior art sunless tanning compositions, and still retain the benefits of the sunless tanning composition. It is sometimes desirable to rinse after application of a sunless tanning composition. In the case of certain prior art sunless tanning compositions, it is expected that people need to wait up to about 12 hours before showering because of the risk of removing the tanning active component. In contrast, it is believed that one can shower much sooner after application of the sunless tanning composition according to the invention without risk of removing all of the tanning active component. It is believed that the polymer component is at least partly responsible for holding the tanning active component to the skin tissue and for providing a sustained release of the tanning active component to the skin tissue.

The polymer component can be prepared from a topical composition precursor that can be prepared by melt processing a hydrophobic polymer composition and a hydrophilic polymer composition to provide an interaction between the hydrophobic polymer composition and the hydrophilic polymer composition. It should be understood that the phrase “melt processing” refers to mixing the hydrophobic polymer composition and the hydrophilic polymer composition under conditions that provide that the hydrophobic polymer component of the hydrophobic polymer composition and the hydrophilic polymer component of the hydrophilic polymer composition are in a liquid state so that they sufficiently mix. When the polymers are sufficiently mixed, it is believed that an interaction forms between the hydrophobic polymer component and the hydrophilic polymer component. The melt processing temperature can be at least about 50° C. and can be at least about 125° C. to generate this interaction.

It is believed the interaction exhibited between the hydrophobic polymer component and the hydrophilic polymer component is a type of complex formation reaction, and that the complexes, once formed, are stable in water at temperatures up to 65° C. and at a pH range of 3.0 to 9.0. By stable, it is meant that the complexes do not favor disassociation under these conditions. It is believed that this interaction provides the sunless tanning composition with an ability to bind or hold onto the tanning active component that may be hydrophobic, allows the sunless tanning composition to be emulsified in water, and provides the sunless tanning composition with an ability to bind to skin. The result of the interaction between the hydrophobic polymer component and the hydrophilic polymer component can be referred to as a hydrophobic polymer/hydrophilic polymer adduct. It should be understood that the term “adduct” is used to refer to the interaction between the hydrophobic polymer component and the hydrophilic polymer component. The interaction may be a form of complexing, but that is only theory. Accordingly, it should be understood that the term “adduct” is not meant to limit the polymer component to a particular theory of interaction.

It is believed that the interaction between the hydrophobic polymer component and the hydrophilic polymer component can be achieved more easily in the absence of water. It is expected that that if the hydrophilic polymer component becomes dissolved in water before forming the complex, it can be more difficult to sufficiently mix the hydrophobic polymer component and the hydrophilic polymer component to provide the desired level of interaction. Although a convenient technique for providing the desired level of interaction between the hydrophobic polymer component and the hydrophilic polymer component is melt mixing, it is expected that other techniques can be used to achieve the desired level of interaction.

The hydrophobic polymer composition that can be used according to the invention includes at least one hydrophobic polymer and can include a mixture of hydrophobic polymers. The hydrophobic polymer composition can include components having repeating pyrrolidone/alkylene groups. Exemplary polymers having repeating pyrrolidone/alkylene groups include poly(vinylpyrrolidone/alkylene) polymers. Poly(vinylpyrrolidone/alkylene) polymers include those polymers obtained by polymerizing alkylene substituted vinylpyrrolidone. Poly(vinylpyrrolidone/alkylene) polymers can be represented by the following general formula: 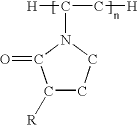
wherein R represents a carbon chain substitute such as an alkylene group and n represents the number of repeating units. The R group is preferably sufficiently long so that the polymer remains relatively water insoluble and should not be too long so that the polymer is difficult to melt process. The alkylene group can contain a length of at least about 10 carbon atoms and can contain no more than about 30 carbon atoms. The alkylene group can contain between about 14 carbon atoms and about 22 carbon atoms, and can contain between about 15 carbon atoms and about 19 carbon atoms.

The poly(vinylpyrrolidone/alkylene) polymers that can be used according to the invention can have a molecular weight that is sufficiently high so that the polymer maintains its water insolubility but the molecular weight should not be so high that it becomes difficult to melt process the polymer. The weight average molecular weight of the poly(vinylpyrrolidone/alkylene) polymer can be between about 3,000 and about 400,000. Another way to characterize the size of the poly(vinylpyrrolidone/alkylene) polymer is by the number of repeating units (n). In the case of a poly(vinylpyrrolidone/alkylene) polymer having a weight average molecular weight of between about 6,000 and about 30,000, the poly(vinylpyrrolidone/alkylene) polymer can have between about 20 and about 80 repeating units, and can have between about 30 and about 50 repeating units. It should be understood that repeating units refer to the residues of vinylpyrrolidone/alkylene groups.

Exemplary poly(vinylpyrrolidone/alkylene) polymers that can be used according to the invention include poly(vinylpyrrolidone/1-eicosene) and poly(vinylpyrrolidone/hexadecene). Poly(vinylpyrrolidone/1-eicosene) can be referred to as PVPE and is commonly used in pharmaceutical and cosmetic preparations. An exemplary form of PVPE for use according to the invention includes about 43 to 44 repeating units in length and has a weight average molecular weight of about 17,000 and can be characterized as a paraffin-like solid. This particular PVPE is highly insoluble in water, and has an extremely low oral toxicity (LD₅₀>17000 mg/kg) and exhibits no demonstrable dermal toxicity. Poly(vinylpyrrolidone/1-hexadecene) can be referred to as PVPH. An exemplary form of PVPH is available as a viscous yellow liquid that is insoluble in water and has a low oral toxicity (LD₅₀>64000 mg/kg), has about 39 to 40 repeating units, a molecular weight of about 14,000, and exhibits no demonstrable dermal toxicity.

PVPE and PVPH differ in the length of the hydrocarbon side chain, and are used extensively in the skin care industry, usually in concentrations of less than 1% by weight, because of their ability to bind to skin. Because the skin care industry generally prefers to apply actives to skin using a water-based composition, the use of PVPE and PVPH often requires solvents, surfactants, and emulsifiers to stabilize these polymers in a water emulsion. However, many of the solvents, surfactants and emulsifiers used to stabilize PVPE and PVPH in a water emulsion lack the low dermal toxicities of PVPE and PVPH. PVPE and PVPH by themselves lack a cosmetically elegant appeal when applied directly to the skin. They tend to be sticky and greasy.

The hydrophobic polymer composition used according to the invention can be provided as a mixture of different poly(vinylpyrrolidone/alkylene) polymers. The mixture of different poly(vinylpyrrolidone/alkylene) polymers can include at least 5 wt. % of a first poly(vinylpyrrolidone/alkylene) polymer based on the weight of the hydrophobic polymer composition. The hydrophobic polymer composition can include between about 5 wt. % and about 54 wt. % of the first poly(vinylpyrrolidone/alkylene) polymer. The second poly(vinylpyrrolidone/alkylene) polymer can be provided in an amount of at least about 46 wt. % and can be in a range of between about 46 wt. % and 95 wt. % based on the weight of the hydrophobic polymer composition. For a hydrophobic polymer composition containing a first poly(vinylpyrrolidone/alkylene) polymer and a second poly(vinylpyrrolidone/alkylene) polymer, the mole ratio of the first polymer to the second polymer can be between about 1:22 and about 1:1. When the hydrophobic polymer composition contains a mixture of different poly(vinylpyrrolidone/alkylene) polymers, the poly(vinylpyrrolidone/alkylene) polymers can be selected to provide improved properties compared to a sunscreen having a hydrophobic polymer composition containing a single poly(vinylpyrrolidone/alkylene) polymer.

When the hydrophobic polymer composition is provided as a mixture of PVPH and PVPE, the PVPH can be provided in a range of between about 46 wt. % to about 95 wt. % and the PVPE can be provided in a range of between about 5 wt. % and about 65 wt. %, based upon the weight of the hydrophobic polymer composition.

The hydrophilic polymer composition that can be used according to the invention includes at least one hydrophilic polymer and may include a mixture of hydrophilic polymers. The hydrophilic polymers that can be used according to the invention include polymers having repeating carboxylic acid groups and/or hydroxyl groups. Exemplary hydrophilic polymers that can be used according to the invention include polyacrylic acid polymers and poly(maleic acid/methylvinylether) copolymers. The hydrophilic polymers should have a molecular weight that is not too high so that the hydrophilic polymer becomes difficult to process.

Polyacrylic acid polymers that can be used according to the invention include those having a weight average molecular weight of at least about 50,000. Polyacrylic acid polymers that can be used include those having a weight average molecular weight between about 50,000 and about 4,000,000. The polyacrylic acid polymers can have a level of cross-linking that is less than about 1%. A general structural representation of polyacrylic acid polymers is shown below: 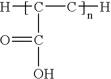
wherein n is the number of repeating units. The number n can be between about 1,000 and about 20,000.

Poly(maleic acid/methylvinylether) copolymers that can be used according to the invention can have a weight average molecular weight of at least about 50,000, and can have a weight average molecular weight of between about 50,000 and about 4,000,000. The weight average molecular weight can be between about 70,000 and 2,500,000. A general structural representation of poly(maleic acid/methylvinylether) copolymers is shown below: 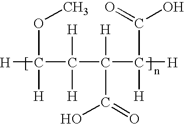
wherein n is the number of repeating units. The number n can be between about 200 and about 20,000.

Additional hydrophilic polymers that can be used according to the invention include starch, derivatives of starch, polyvinyl alcohol, cellulose, derivatives of cellulose, carboxymethyl cellulose, cyclodextrins, and dextrans. The weight average molecular weight of the hydrophilic polymers is preferably sufficient to provide solubility in water but not too high to become difficult to process. Exemplary starches include amylopectin and polyglucose. Starches that can be used according to the invention can have a weight average molecular weight of about 50,000 to about 20,000,000. A derivative of starch that can be used according to the invention includes partially hydrolyzed starch. Cellulose that can be used according to the invention can have a weight average molecular weight of about 50,000 to about 15,000,000. Polyglucose that can be used according to the invention can be characterized as low fraction polyglucose having a weight average molecular weight of about 60,000 to about 90,000, and high fraction polyglucose having a weight average molecular weight of about 90,000 to about 300,000. An exemplary low fraction polyglucose material that can be used according to the invention is available under the name Dextran-70. In general, this type of polyglucose has all alpha 1-6 linkages. Starch derivatives that can be used according to the invention include those starch derivatives having alpha 1-4 linkages. An example of this type of starch derivative includes cyclodextrins. Exemplary cyclodextrins that can be used according to the invention include those that act to provide a cavity within the molecule large enough to contain components desirable for topical applications. Cyclodextrins that can be used according to the invention can have a molecular weight of about 900 to about 1,400. Polyvinyl alcohols that can be used according to the invention include those with a weight average molecular weight of about 50,000 to about 200,000.

Exemplary hydrophilic polymers that can be used according to the invention include those polymers having the following melting temperature range and the following maximum temperature range beyond which it is expected decomposition of the polymer will occur. Exemplary poly(maleic acid/methylvinylether) copolymers that can be used include those having a melting temperature range of about 60° C. to about 65° C. and a maximum temperature range of about 80° C. to about 90° C. Exemplary polyacrylic acid polymers that can be used include those having a melting temperature range of about 65° C. to about 70° C. and a maximum temperature range of about 80° C. to about 90° C. Exemplary carboxymethyl cellulose polymers that can be used include those having a melting temperature range of about 55° C. to about 60° C. and a maximum temperature range of about 75° C. to about 80° C. Exemplary polyvinyl alcohol polymers that can be used include those having a melting temperature range of about 50° C. to about 55° C. and a maximum temperature range of about 65° C. to about 70° C. Exemplary starches that can be used include those having a melting temperature range of about 40° C. to about 45° C. and a maximum temperature range of about 50° C. to about 55° C. Exemplary dextrans that can be used include those having a melting temperature range of about 37° C. to about 40° C. and a maximum temperature range of about 45° C. to about 50° C. Exemplary β-cyclodextrins that can be used according to the invention include those having a melting temperature range of about 40° C. to about 45° C. and a maximum temperature range of about 65° C. to about 70° C.

The hydrophobic polymer composition and the hydrophilic polymer composition can be combined and heated to at least about 50° C. to provide a polymer melt. The composition can be heated to at least about 125° C. under mixing to form complexes between the hydrophobic and hydrophilic polymers. It should be understood that a polymer melt refers to a polymer that flows or becomes a liquid when heated and is not meant to refer to a polymer that forms a liquid as a result of being dissolved in a solvent.

The complex formation step can be carried out in a relatively anhydrous environment. That is, the amount of water provided in the composition during the complex formation step can be less than about 1 wt. %. Once the desired level of complex formation has occurred, the composition can be hydrated with water.

The hydrophobic polymer composition and the hydrophilic polymer composition can be mixed together in amounts sufficient to provide a ratio of pyrrolidone groups to the combination of carboxylic acid groups and hydroxyl groups of about 1:1 to about 5:1. The ratio of the structures causing the observed interaction between the hydrophobic polymer composition and the hydrophilic polymer composition can be referred to as “functional group parity.” The ratio of pyrrolidone groups to the combination of carboxylic acid groups and hydroxyl groups can be about 1.5:1 to about 3:1. In order to drive the complex formation reaction, it is desirable to provide an imbalance between the two types of groups. Accordingly, it is generally desirable to provide more of the pyrrolidone groups than the combination of carboxylic groups and the hydroxyl groups. It should be understood that the reference to a “combination of carboxylic groups and hydroxyl groups” refers to the total amount of carboxylic groups and hydroxyl groups present but does not require the presence of both carboxylic groups and hydroxyl groups. For example, the value of the combination of carboxylic groups and hydroxyl groups can be determined for a composition that contains only carboxylic groups. Similarly, the value can be determined for a composition that contains only hydroxyl groups.

During the complex formation step, the amounts of hydrophobic polymer composition and hydrophilic polymer composition can be characterized on a weight percent basis. For example, about 2 wt. % to about 28 wt. % hydrophilic polymer composition and about 72 wt. % to about 98 wt. % hydrophobic polymer composition can be combined to provide for complex formation. About 8 wt. % to about 25 wt. % hydrophilic polymer composition and about 72 wt. % to about 95 wt. % hydrophobic polymer composition can be combined to form the complex. During the complex formation step, the amount of water available in the composition can be less than about 1 wt. %. Although the complex forming composition can be relatively anhydrous, it is expected that the amount of water will be between about 0.3 wt. % and about 1.0 wt. %.

Once the hydrophobic polymers and the hydrophilic polymers have sufficiently reacted or interacted to form a complex, water can be added to the composition to provide a stable aqueous composition that can be relatively easily further hydrated. It has been found that the first hydration of the topical composition precursor is the most difficult hydration step because of the need to control the conditions of hydration. After the first hydration to a water content of at least about 30 wt. %, it is expected that further hydrations to higher water contents are relatively easy and can be accomplished by simply mixing the composition with water. Accordingly, the amount of water provided in the composition when made available as a concentrate for shipment is preferably between about 30 wt. % and about 45 wt. %. When the composition includes about 30 wt. % to about 45 wt. % water, it is expected that the composition will include between about 3 wt. % and about 10 wt. % hydrophilic polymer composition and between about 30 wt. % and about 50 wt. % hydrophobic polymer composition.

Water can be added to the relatively anhydrous composition by mixing water and the relatively anhydrous composition at a temperature and for a time sufficient to allow the composition to become hydrated without losing significant amounts of interaction between the hydrophobic polymer composition and the hydrophilic polymer composition. The relatively anhydrous composition can be hydrated by heating to at least 60° C. and adding water while mixing. The composition can be heated to at least about 65° C. and to at least about 70° C. An exemplary temperature range is about 65° C. to about 80° C.

The relatively anhydrous composition can be referred to as the topical composition precursor and generally refers to the hydrophobic polymer/hydrophilic polymer adduct. The polymer component for the sunless tanning composition can refer to a composition that contains only the hydrophobic polymer/hydrophilic polymer adduct, and it can refer to a composition wherein the hydrophobic polymer/hydrophilic polymer adduct is diluted with water. In general, it is desirable to have a sufficient amount of water in the polymer component that allows one to formulate the polymer component into the sunless tanning composition according to the invention. If there is too little water in the polymer component, it may become difficult to formulate the sunless tanning composition. For example, the polymer component can contain water in an amount of up to about 95 wt. %. The polymer component can have a water concentration of between about 30 wt. % to about 45 wt. %.

Additional components can be added to the skin bonding polymer composition. For example, it may be desirable to add a component that helps stabilize the hydrophobic polymer/hydrophilic polymer adduct, and to help preserve and/or maintain the composition.

Tanning Active Component

The sunless tanning composition includes a tanning active component so that when the sunless tanning composition is applied to skin tissue, the skin tissue browns or darkens to simulate a tanning effect. The tanning active component can be referred to as a “sunless tanning active,” a “skin tanner,” or a “self tanning agent.” These terms can be used interchangeably. The tanning active component refers to any compound or material that imparts a tan or brown appearance to skin tissue by exposure thereto without the need to expose the skin tissue to sun or other radiant energy source. Exemplary tanning active components include dihydroxyacetone (DHA), 1,3,4-trihydroxy-2-butanone (available under the name Erythrulose from Penta Pharm of Switzerland), botanical extracts of plants such as the silver birch (Betulla alba), or Eclipta alba which contain flavonoids known as wedelolactone, dimethylwedelolacetone and mixtures thereof. Such botanical sources are described in U.S. Pat. No. 5,559,146. One particular formulation is known as Mahakanni STLC, a self-tanning liposome concentrate whose active ingredients are believed to be derived from Eclipta alba, as described in Faking It Without DHA, Soap, Perfumery and Cosmetics, September 1996, pp. 33-35. Such botanical extracts can be used alone or in combination with DHA. It should be understood that DHA can be considered a preferred tanning active component according to the present invention.

The tanning active component can be included in the sunless tanning composition in an amount to provide a desired level of skin darkening effect. It is believed that the polymer component helps provide a sustained release of the tanning active component so that lower levels of tanning active component can be used to provide the desired effect. In addition, certain tanning active components may be considered irritating to the skin at certain higher levels, and it is believed that the polymer component may allow use of higher amounts of the tanning active component without skin irritation for many individuals. In general, the tanning active component can be included in the sunless tanning composition in an amount of at least about 0.5 wt. % and can be provided at an amount of less than about 18 wt. %. In the case of the use of dihydroxyacetone (DHA) as the tanning active component, it can be used in an amount of about 2 wt. % to about 18 wt. %, and can be used in an amount of about 4 wt. % to about 15 wt. %. In the case of 1,3,4-trihydroxy-2-butanone, it can be used in an amount of about 0.5 wt. % to about 3 wt. %.

In general, it is expected that it is desirable for the tanning active component to remain in contact with the skin for at least 4 hours and preferably at least 12 hours to allow for completion of the darkening effect. It is believed that the polymer component helps keep the tanning active component proximate the skin tissue even after rinsing with water in a shower.

pH Adjusting Agent

The sunless tanning composition can include a pH adjusting agent to provide the sunless tanning composition with a pH that helps stabilize the tanning active component. By maintaining certain tanning active components, such as dihydroxyacetone (DHA) or 1,3,4-trihydroxy-2-butanone, in a composition at a pH below about 5, the stability of the tanning active component can be maintained and the tanning active component can remain available for providing a darkening effect when exposed to skin tissue. Exemplary pH adjusting agents that can be used include citric acid, lactic acid, acetic acid, propionic acid, and mixtures thereof.

The sunless tanning composition can include a pH adjusting agent in an amount sufficient to provide the composition with a pH sufficient to preserve the stability of the tanning active component. In addition, the pH of the sunless tanning composition should not be so low that the sunless tanning composition irritates and/or burns skin tissue. For example, the pH adjusting agent can be provided in the sunless tanning composition in an amount sufficient to provide the composition with a pH of about 3 to about 5, and preferably about 3.5 to about 4.5. In the case of citric acid as the pH adjusting agent, the amount of citric acid can be provided at a level of about 0.1 wt. % to about 0.3 wt. % to provide the desired pH.

The polymer component of the lotion may be at least in part responsible for reducing the irritability of the sunless tanning composition at low pH values. For example, it is believed that the polymer component may help reduce irritation of skin tissue resulting from the low pH and resulting from the tanning active component.

Water

The sunless tanning composition can include water in an amount sufficient to allow the composition to be applied to skin tissue while providing the desired coverage of the tanning active component over the skin tissue. The water component can be provided as deionized water, filtered water, distilled water, reverse osmosis water, or tap water. In the event that the water includes hardness or other components, it may be desirable to include builders, sequestrants, and chelating agents to handle the water hardness. In general, the sunless tanning composition can include at least about 50 wt. % water. In addition, it is expected that if there is too much water, the emulsion might become unstable. In general, the amount of water in the sunless tanning composition can be less than about 95 wt. %. The amount of water in the sunless tanning composition can be about 65 wt. % to about 93 wt. %.

Surfactant Component

The sunless tanning composition can include a surfactant component to help maintain the composition as an emulsion. In general, an emulsion refers to a composition that resists phase separation after sitting at room temperature for a couple of months. In general, it is expected that the sunless tanning composition can be stored in a warehouse or in a storage closet for at least two months and can remain as an emulsion during that two month period. Preferably, the sunless tanning composition can remain as an emulsion for at least one year or at least two years. The ability of the sunless tanning composition to remain as an emulsion can be tested according to an accelerated stability test where the composition is held at 45° C. for two months. It is expected that this accelerated stability test for two months roughly corresponds to a period of about two years at room temperature. In general, it is expected that the sunless tanning composition will remain as an emulsion after sitting for one month at 45° C. and preferably at least two months at 45° C.

Exemplary surfactants that can be used as the surfactant component include nonionic surfactants that help stabilize the emulsion, wet skin tissue, and provide a generally even distribution of the tanning active component. Exemplary nonionic surfactants that can be used include glycerol stearate such as glycerol monostearate, polysorbate such as that available under the name Tween 60, and polyoxyethylene stearate. In addition, mixtures of nonionic surfactants can be included including mixtures of polysorbate and glycerol stearate.

It is believed that anionic surfactants may be useful as part of the surfactant component. In general, it is expected that anionic surfactants have a greater tendency to cause irritation to skin tissue.

The sunless tanning composition can include an amount of surfactant component sufficient to provide the composition with a desired emulsion stability and sufficiently low viscosity without foaming. The amount of the surfactant component in the sunless tanning composition, if present at all, can be about 1 wt. % to about 6 wt. %, and can be about 2 wt. % to about 5 wt. %.

Thickener

Thickeners that can be incorporated into the sunless tanning composition include those components that thicken or increase the viscosity of the sunless tanning composition so that the sunless tanning composition can be readily applied to skin. Thickeners that can be used in the sunless tanning composition include those components often referred to as viscosity controlling agents.

Exemplary thickeners or viscosity controlling agents that can be provided in the sunless tanning composition include cellulose gum, alkane triols; acrylates; substituted celluloses such as methylcellulose, and hydroxypropyl cellulose; cetyl alcohol; gums such as natural gums or synthetic gums; long chain alcohols such as those having about 9 to about 24 carbon atoms; polyglycols such as polyethylene glycols, polypropylene glycols, polybutylene glycols, polyethylene propylene glycols, or mixtures thereof; waxes such as natural waxes or synthetic waxes; hydrogenated oils; glycol esters; fatty acid esters; long chain acids; acid amides; silicates; and mixtures thereof. An exemplary thickener that can be used is hydroxyethyl cellulose.

The sunless tanning composition may or may not include a thickener. When the sunless tanning composition includes a thickener, the thickener can be provided in an amount that provides the desired level of thickening. The sunless tanning composition can include a thickener in an amount of least about 0.1 wt. % and can include a thickener in an amount of at least about 0.4 wt. %. In addition, the thickener can be provided in an amount of less than about 2 wt. %, and can be provided in an amount of less than about 1.0 wt. %.

Emollient

The sunless tanning composition can include an emollient for improving the texture of the composition. An emollient is an oleaginous or oily substance which helps to smooth and soften the skin, and may also reduce its roughness, cracking or irritation. Typical suitable emollients include mineral oil, having a viscosity in the range of 50 to 500 centipoise (cps), lanolin oil, coconut oil, cocoa butter, olive oil, almond oil, macadamia nut oil, synthetic jojoba oils, natural sonora jojoba oils, safflower oil, corn oil, liquid lanolin, cottonseed oil and peanut oil.

Other suitable emollients include squalane, castor oil, polybutene, odorless mineral spirits, sweet almond oil, avocado oil, clophyllum oil, ricin oil, vitamin E acetate, olive oil, linolenic alcohol, oleyl alcohol, the oil of cereal germs such as the oil of wheat germ, isopropyl palmitate, octyl palmitate which is commercially available as Lexol EHP, tradename of Inolex Co. of Philadelphia, Pa., isopropyl myristate, hexadecyl stearate, butyl stearate, decyl oleate, acetyl glycerides, the octanoates and benzoates of (C₁₂-C₁₅) alcohols, the octanoates and decanoates of alcohols and polyalcohols such as those of glycol and glycerol, ricin oleates of alcohols and poly alcohols such as those of isopropyl adipate, hexyl laurate and octyl dodecanoate.

Other suitable emollients which are solids or semi-solids at room or ambient temperatures may be used in amounts sufficient to provide liquid topical compositions. Such solid or semi-solid cosmetic emollients include hydrogenated lanolin, hydroxylated lanolin, acetylated lanolin, petrolatum, isopropyl lanolate, butyl myristate, cetyl myristate, myristyl myrislate, myristyl lactate, cetyl alcohol, isostearyl alcohol and isocetyl lanolate. Exemplary emollients include stearic acid, cetyl alcohol, natural and synthetic esters such as coconut oil. The sunless tanning composition can include the emollient in an amount sufficient to provide a silky feel. An exemplary range of the emollient in the sunless tanning composition can be at least about 0.5 wt. %. In addition, the sunless tanning composition can include an emollient in an amount of less than about 3 wt. %. It should be understood that the emollient is an optional component of the sunless tanning composition.

Moisturizer

The sunless tanning composition can include a moisturizer to provide a desired moisturizing effect to skin tissue. The moisturizer can be provided as a humectant. In general, a humectant is a moistening agent that promotes retention of water due to its hydroscopic properties. Exemplary humectants include glycerine, polymeric glycols such as polyethylene glycol and polypropylene glycol, and sorbitols such as sorbitol solution, pyrrolidone carboxylic acid, urea, or mixtures thereof. The sunless tanning composition can be provided without a moisturizer. When the sunless tanning composition includes a moisturizer, it can be included in an amount of at least about 0.5 wt. %. In addition, the sunless tanning composition can include a moisturizer in an amount of less than about 5 wt. %.

Coloring Agent

The sunless tanning composition can, if desired, include a colorant or coloring agent such as a dye, pigment, or tint. In general, it is expected that a coloring agent may be useful to help gauge application of an even coating of the sunless tanning composition to skin tissue. In addition, the coloring agent can be used to provide the user of the sunless tanning composition with a sense that a darkening of the skin has occurred even before the tanning active component has had a chance to provide a darkening effect.

Exemplary coloring agents include certified dyes that are synthetic organic coal tar derivatives which are manufactured so that each batch passes a Food & Drug Administration (FDA) purity inspection. If approved by the FDA, these dyes are certified for use in foods, drugs, cosmetics (FDA colors), drugs and foods only (DC colors), or in topically applied drugs and cosmetics (External DC colors). Certified dyes can be water soluble or lakes. Lakes are organic pigments prepared by precipitating a soluble dye on a reactive or absorbent stratum which is an essential part of the pigment's composition. Most lakes are aluminum, barium or calcium derived. These insoluble pigments are used mostly in makeup products, either powders or liquids, when a temporary color is desired that will not stain the skin (as oil-soluble dyes tend to do). The lakes are used in these products along with inorganic colors such as iron oxide, zinc oxide, and titanium dioxide (the whitest white pigment).

Water soluble, certified dyes are used mostly in color products, not skin or hair, although it is possible to make a temporary hair color rinse using only certified dyes. When incorporating these dyes in an emulsion, they will be soluble in the external water phase in an oil/water system. It is useful to know the solubility properties of the certified dyes in various solvents and their stability to reactive chemicals. Table I lists some of the currently available water soluble certified dyes.

TABLE I

Water-Soluble Dyes
FDC Blue #1
FDC Blue #2
FDC Green #3
FDC Red #3
FDC Red #40
FDC Yellow #5
FDC Yellow #6
DC Green #5
DC Red #22
DC Red #28
DC Red #33
DC Yellow #10
Ext DC Violet #2
Ext DC Yellow #7
DC Green #8
DC Orange #4
DC Yellow #8

The water-soluble color dye can also be a natural color such as caramel color or walnut see extract color.

The sunless tanning composition can contain the water-soluble color dye (color indicator) in an amount sufficient to enable the composition to be readily visualized (i.e. colored) when initially applied to the skin, such that when the emulsion dries after being spread on the skin and/or is rubbed out using one's hand and/or fingers, the color substantially disappears.

If the sunless tanning composition includes a coloring agent, it can be included in an amount sufficient to provide the desired amount of coloring. One or more coloring agents can be used in the composition in an amount of about 0.00001 to about 0.5% by weight of the composition. In addition, it can be used in an amount of about 0.0001 to about 0.2%, and in an amount of about 0.001 to about 0.1%. It should be understood that the sunless tanning composition can be provided without a coloring agent.

Preservatives

The sunless tanning composition can include preservatives for prevention of bacterial, fungal, and/or yeast contamination. Exemplary preservatives that can be used in the sunless tanning composition include phenoxyethanol, benzoic acid, derivatives and salts of benzoic acid, parabens, oxazolidines, chlorinated aromatic compounds and phenols, hydantoins, cresols and derivatives, imiazolindinyl urea, iodopropanol butylcarbamate, sulfites, and bisulfites. The sunless tanning composition can include any of the preservatives commonly used or known to be suitable for topically applied compositions.

The sunless tanning composition can be formulated without a preservative. It is expected that the preservative will increase the shelf life of the sunless tanning composition by reducing or preventing the growth of bacteria, fungus, and/or yeast. When the sunless tanning composition includes a preservative, the preservative is preferably provided in an amount sufficient to provide a desired level of protection from growth of bacteria, fungus, and/or yeast. In general, for most preservatives, it is expected that the amount of preservative will be provided at a level of between about 0.25 wt. % and about 1.0 wt. %, and can be provided at a level of between about 0.3 wt. % and about 0.5 wt. %, based on the weight of the sunless tanning composition.

Antioxidants

The sunless tanning composition can include antioxidants to help increase the shelf life of the sunless tanning composition by reducing oxidation of the skin coloring agent. Exemplary antioxidants that can be used include vitamins such as vitamin E, vitamin E acetate, vitamin C, vitamin A, and vitamin D, and derivatives thereof. Exemplary antioxidants include α-tocopherols which can be characterized as natural or synthetic Vitamin E. Additional exemplary antioxidants include propyl, octyl and dodecyl esters of gallic acid, butylated hydroxyanisole (BHA)(usually as a mixture of ortho and meta isomers), butylated hydroxytoluene (BHT), and nordihydroguaiaretic acid, and alkylated parabens such as methylparaben and propylparaben.

The sunless tanning composition can be formulated without an antioxidant. When the sunless tanning composition includes an antioxidant, the antioxidant is preferably provided in an amount that provides antioxidant properties in the sunless tanning composition. In general, it is expect that the antioxidant can be provided in an amount of between about 0.2 wt. % and about 2 wt. %, and can be provided in an amount of between about 0.7 wt. % and about 1.5 wt. %, based on the weight of the sunless tanning composition. In the case of vitamin E, it is expected that the vitamin E can be included in the sunless tanning composition in an amount of about 0.1 wt. % to about 1 wt. %, and can be included in an amount of about 0.3 wt. % to about 0.8 wt. %.

One or more antioxidants can optionally be included in the emulsion in an amount ranging from about 0.001 to about 5 weight percent, preferably about 0.05 to about 2 percent. It should be appreciated that the sunless tanning composition can be provided without an antioxidant.

Chelating Agents

Chelating agents are substances used to chelate or bind metallic ions with a certain heterocyclic ring structure so that the ion is held by chemical bonds from each of the participating rings. Suitable chelating agents include ethylene diaminetetraacetic acid (EDTA), EDTA trisodium, EDTA tetrasodium, calcium disodium edetate, EDTA trisodium, EDTA tetrasodium and EDTA dipotassium. One or more chelating agents can optionally be included in the emulsion in amounts ranging from about 0.001 to about 0.1 weight percent. It should be appreciated that the sunless tanning composition can be provided without a chelating agent.

Fragrances

Fragrances are aromatic compounds which can impart an aesthetically pleasing aroma to the sunless tanning emulsion. Typical fragrances include aromatic materials extracted from botanical sources (i.e. rose petals, gardenia blossoms, jasmine flowers, etc.) which can be used alone or in any combination to create essential oils. Alternatively, alcoholic extracts may be prepared for compounding fragrances. One or more fragrances can optionally be included in the composition in an amount ranging from about 0.001 to about 10 weight percent, preferably about 0.05 to about 5 percent. It should be appreciated that the sunless tanning composition can be provided without a fragrance.

Another type of component that may be considered an aesthetic agent or, in some cases, a fragrance, can be referred to as an odor counteractant. In general, an odor counteractant can be used to render the sunless tanning composition relatively odorless. An exemplary odor counteractant that can be used is available under the name Ordenone from Bell Aire Fragrances. If the composition includes an odor counteractant, it can be included in an amount sufficient to counteract certain odors. For example, the composition can include at least about 0.05 wt. % of odor counteractant, can include about 0.1 wt. % to about 1 wt. % odor counteractant, and can include about 0.3 wt. % to about 0.8 wt. % odor counteractant.

The odor counteractant can be used to counteract the odor resulting from certain components such as dihydroxyacetone (DHA).

Stability

The components of the sunless tanning composition can be selected to provide desired stability of the composition. In general, stability can refer to the ability of the composition to remain as an emulsion over time. In addition, stability can refer to the lack of degradation of the tanning active component. The applicants have found that certain components that have been present in prior art sunless tanning compositions can have an effect of degrading certain tanning active components such as dihydroxyacetone.

The sunless tanning composition can be subjected to an accelerated stability test where the composition is held at 45° C. for one month, and for two months. In general, it is expected that an accelerated stability testing for one month corresponds to a period of about one year at room temperature, and an accelerated stability test for about two months corresponds to about two years at room temperature. In general, after storage at 45° C. for two months, the activity of the tanning active component can be at least 50% of its original level prior to storage at 45° C. for two months. Preferably, the tanning active component can be provided at a level of at least about 75% of its original level prior to testing, and more preferably at least about 90% of its original level prior to testing. By way of example, a sunless tanning composition containing dihydroxyacetone at a level of about 9 wt. % can be subjected to storage at 45° C. for two months, and the resulting composition can have a level of active dihydroxyacetone of at least about 4.5 wt. %. Preferably, after being subjected to storage at 45° C. for two months, the resulting composition can have a level of dihydroxyacetone of at least about 6.75 wt. %, and more preferably at least about 8 wt. %.

It should be understood that certain components can, if desired, be excluded from the sunless tanning composition. For example, certain components that have a tendency to react with the tanning active component can be excluded. Exemplary types of materials that can be excluded include amines such as triethanolamine and protein components such as aloe vera. In addition, other components having free carboxyl groups can be minimized because of their tendency to react with certain tanning active components. In addition, because of the presence of the polymer component, other components found in prior art sunless tanning compositions can be reduced or excluded from the sunless tanning composition according to the invention, if desired. These additional components include for example emollients, humectants, dry-feel modifiers, alcohols, and silicones. Although these components can be excluded from the sunless tanning composition according to the invention, they can additionally be included in any desired amounts. When excluded, they can be either totally excluded or excluded in amounts greater than about 0.1 wt. % for each of the components.

It should be understood that the sunless tanning composition can be prepared by mixing the components together to provide an emulsion that preferably resists phase separation over a period of at least two months.

### EXAMPLE

Two sunless tanning lotions were subjected to accelerated stability testing where the lotions, provided in containers, were stored at 45° C. for two to three months. The lotions are reported in Tables 1 and 2. Comparative composition A is a lotion not according to the invention, and composition B is a lotion according to the invention.

TABLE 1
Comparative Composition A

| Component | Wt. % |
|---|---|
| Water | 72.45 |
| Polymer A | 6.0 |
| Dihydroxyacetone | 9.0 |
| Soy Bean Oil | 3.0 |
| Glycerin | 2.50 |
| Cetyl Alcohol | 2.0 |
| Coconut Oil | 2.0 |
| Glyceryl Stearate | 1.0 |
| Triethanolamine | 1.00 |
| Aloe 100:1 | 0.20 |
| Carbomer | 0.25 |
| Phenoxyethanol | 0.50 |
| Tocopherol (Vitamin E) | 0.10 |

Comparative Composition A had a pH of 5.5. Polymer A is a skin bonding polymer component prepared from maleic acid methylvinylether copolymer and a mixture of polyvinylpyrrolidone/1-hexadecene and polyvinylpyrrolidone/1-eicosene.

TABLE 2
Comparative Composition B

| Component | Wt. % |
|---|---|
| Water | 77.40 |
| Polymer B | 6.00 |
| Dihydroxyacetone | 9.00 |
| Glyceryl Stearate | 3.00 |
| Hydroxyethylcellulose | 0.40 |
| Phenoxyethanol | 0.50 |
| Glycerin | 1.00 |
| Citric Acid | 0.20 |
| Stearic Acid | 1.00 |
| Ordenone | 0.50 |
| Tween-60 | 1.00 |

Composition B exhibited a pH of 4.2. Polymer B was prepared from starch and a mixture of polyvinylpyrrolidone/1-hexadecene and polyvinylpyrrolidone/1-eicosene.

The results of the accelerated stability testing is reported in Table 3. The results report the weight percent of DHA (dihydroxyacetone) remaining in the composition after days of testing.

TABLE 3
Accelerated Stability Testing Results

| Days | 0 | 14 | 21 | 28 | 35 | 56 | 84 | 112 |
|---|---|---|---|---|---|---|---|---|
| Comparative Composition A | 9.0 | 7.4 | 6.2 | 4.1 | 2.9 | | | |
| (wt. % DHA) | | | | | | | | |
| Comparative Composition B | 9.0 | 9.0 | | 8.9 | | 8.8 | 8.6 | 8.1 |
| wt. % DHA) | | | | | | | | |

The above specification, examples and data provide a complete description of the manufacture and use of the composition of the invention. Since many embodiments of the invention can be made without departing from the spirit and scope of the invention, the invention resides in the claims hereinafter appended.

We claim:

1. A sunless tanning composition comprising:
(a) an effective amount of a tanning active component to provide a darkening effect of skin tissue after exposure to the skin tissue;
(b) an effective amount of a skin bonding polymer component to expose the tanning active component to skin tissue, when applied to skin tissue, so that the tanning active component provides a darkening effect to the skin tissue;
(c) water;
(d) an effective amount of an acidifying agent to provide the composition with a pH of less than about 5; and
(e) wherein the composition has a viscosity of at least about 3,000 cSt.

2. A sunless tanning composition according to claim 1, wherein the tanning active component, after storage at 45° C. for two months, remains at a level of at least about 50% of the level of the tanning active component prior to storage at 45° C. for two months.

3. A sunless tanning composition according to claim 1, wherein the composition comprises at least about 50 wt. % water.

4. A sunless tanning composition according to claim 1, wherein the composition comprises about 65 wt. % to about 95 wt. % water.

5. A sunless tanning composition according to claim 1, wherein the composition comprises a thickening agent.

6. A sunless tanning composition according to claim 1, wherein the composition comprises about 0.4 wt. % to about 1 wt. % of the thickening agent.

7. A sunless tanning composition according to claim 5, wherein the thickening agent comprises a cellulose gum.

8. A sunless tanning composition according to claim 5, wherein the thickening agent comprises at least one of hydroxymethyl cellulose, hydroxyethyl cellulose or hydroxypropyl cellulose.

9. A sunless tanning composition according to claim 1, further comprising a moisturizer.

10. A sunless tanning composition according to claim 9, wherein the composition comprises about 0.5 wt. % to about 5 wt. % of the moisturizer.

11. A sunless tanning composition according to claim 9, wherein the moisturizer comprises at least one of glycerine, pyrrolidone carboxylic acid or urea.

12. A sunless tanning composition according to claim 1, wherein the pH adjusting agent comprises at least one of citric acid, lactic acid, acetic acid, or propionic acid.

13. A sunless tanning composition according to claim 1, wherein the composition comprises about 0.5 wt. % to about 3 wt. % of the pH adjusting agent.

14. A sunless tanning composition according to claim 1, wherein the composition has a pH of less than about 4.5.

15. A sunless tanning composition according to claim 1, wherein the composition comprises about 4 wt. % to about 15 wt. % of the tanning active component.

16. A sunless tanning composition according to claim 1, wherein the tanning active component comprises dihydroxyacetone.

17. A sunless tanning composition according to claim 1, wherein the tanning active component comprises 1,3,4-trihydroxy-2-butanone.

18. A sunless tanning composition according to claim 15, wherein the composition comprises about 0.5 wt. % to about 3 wt. % of the 1,3,4-trihydroxy-2-butanone.

19. A sunless tanning composition according to claim 1, further comprising a surfactant.

20. A sunless tanning composition according to claim 19, wherein the composition comprises about 0.5 wt. % to about 4 wt. % surfactant.

21. A sunless tanning composition according to claim 20, wherein the surfactant comprises a nonionic surfactant.

22. A sunless tanning composition according to claim 1, further comprising about 0.25 wt. % to about 1 wt. % preservative.

23. A sunless tanning composition according to claim 1, wherein the composition comprises about 3 wt. % to about 8 wt. % of the skin bonding polymer component.

24. A sunless tanning composition according to claim 1, wherein the skin bonding polymer component comprises a hydrophobic polymer/hydrophilic polymer adduct.

25. A sunless tanning composition according to claim 1, wherein the skin bonding polymer component comprises an adduct of a hydrophobic polymer composition containing repeating pyrrolidone/alkylene groups and a hydrophilic polymer composition comprising repeating carboxylic acid groups and/or hydroxyl groups.

26. A sunless tanning composition according to claim 25, wherein the hydrophobic polymer composition comprises a poly(vinylpyrrolidone/alkylene) polymer having the following formula: 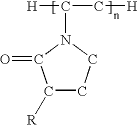
wherein R comprises at least about 10 carbon atoms and N is sufficient to provide a weight average molecular weight of about 3,000 to about 400,000.

27. A sunless tanning composition according to claim 25, wherein the hydrophilic polymer composition comprises starch.

28. A sunless tanning composition according to claim 27, wherein the starch has a weight average molecular weight of about 50,000 to about 20 million.

29. A sunless tanning composition according to claim 27, wherein the starch comprises partially hydrolyzed starch.

30. A sunless tanning composition according to claim 1, further comprising a coloring agent.

31. A sunless tanning composition according to claim 30, wherein the coloring agent comprises at least one certified dye.

32. A sunless tanning composition according to claim 1, wherein the composition comprises vitamin E.

33. A sunless tanning composition according to claim 32, wherein the composition comprises about 0.1 wt. % to about 1 wt. % of the vitamin E.

34. A sunless tanning composition according to claim 1, wherein the composition comprises about 0.1 wt. % to about 1 wt. % of an aesthetic agent.

35. A sunless tanning composition according to claim 34, wherein the aesthetic agent comprises a fragrance.

36. A sunless tanning composition comprising:
(a) an effective amount of a tanning active component to provide darkening of skin tissue when applied to skin tissue;
(b) a polymer component having an average molecular weight of about 2,000 to about 500,000;
(c) water;
(d) an effective amount of an acidifying agent to provide the composition with a pH of less than about 5; and
(e) wherein the composition has a viscosity of at least about 3,000 cSt.

37. A sunless tanning composition according to claim 36, wherein the tanning active component, after storage at 45° C. for two months, remains at a level of at least about 50% of the level of the tanning active component prior to storage at 45° C. for two months.

38. A method of applying a sunless tanning composition to skin tissue comprising:
(a) rubbing a sunless tanning composition having a viscosity of at least about 3,000 cSt onto skin tissue, the sunless tanning composition comprising:
(i) an effective amount of a tanning active component to provide a darkening effect of skin tissue after exposure to the skin tissue;
(ii) an effective amount of a skin bonding polymer component to expose the tanning active component to the skin tissue so that the tanning active component provides a darkening effect to the skin tissue;
(iii) an effective amount of an acidifying agent to provide the composition with a pH of less than about 5; and
(iv) wherein the composition has a viscosity of at least about 3,000 cSt.

39. A method according to claim 38, further comprising:
(a) allowing the sunless tanning composition to remain on the skin tissue for at least about 4 hours.
